(19) United States
(12) Patent Application Publication
(10) Pub. No.: US 2015/0299151 A1
(43) Pub. Date: Oct. 22, 2015

(54) POLYMORPH FORMS OF DESAZADESFERRITHIOCIN ANALOGS

(71) Applicant: FEEROKIN BIOSCIENCES, INC., Wayne, PA (US)

(72) Inventors: Amy E. Tapper, San Carlos, CA (US); Hugh Y. Rienhoff, JR., San Carlos, CA (US); Stephan D. Parent, San Carlos, CA (US); Patricia Andres, San Carlos, CA (US)

(73) Assignee: FerroKin BioSciences, Inc., Wayne, PA (US)

(21) Appl. No.: 14/647,485

(22) PCT Filed: Dec. 6, 2013
(86) PCT No.: PCT/US13/73662

§ 371 Date: May 27, 2015

Related U.S. Application Data
(60) Provisional application No. 61/734,569, filed on Dec. 7, 2012.

(51) Int. Cl. C07D 277/12

(52) U.S. Cl. CPC C07D 277/12; C07B 2200/13

(57) ABSTRACT

The present invention provides a solid form and compositions thereof, which are useful as metal chelators and which exhibit desirable characteristics for the same.

### CROSS REFERENCE TO RELATED APPLICATIONS

This application claims priority to U.S. provisional patent application No. 61/734,569, filed Dec. 7, 2012, the entire contents of which are hereby incorporated by reference herein.

### BACKGROUND OF THE INVENTION

Metal ions are critical to the proper functioning of living systems. Ions such as Fe²⁺, Zn²⁺, Cu²⁺, Ca²⁺, and Co³⁺, to name but a few, can be found in the active sites of over a third of known enzymes and other functional proteins such as RNA polymerase, DNA transcription factors, cytochromes P₄₅₀s, hemoglobin, myoglobin, and coenzymes such as vitamin B₁₂. Therefore, these metals serve to regulate oxidation and reduction reactions, stabilize or shield charge distributions, and orient substrates for reactions.

However, the body has a limited ability to absorb and excrete metals, and an excess can lead to toxicity. As one example, an excess of iron, whether derived from red blood cells chronically transfused, necessary in such conditions such as beta thalassemia major, or from increased absorption of dietary iron such as hereditary hemochromatosis can be toxic through the generation by iron of reactive oxygen species from H₂O₂. In the presence of Fe²⁺, H₂O₂ is reduced to the hydroxyl radical (HO.), a highly reactive species, a process known as the Fenton reaction. The hydroxyl radical reacts very quickly with a variety of cellular constituents and can initiate free radicals and radical-mediated chain processes that damage DNA and membranes, as well as produce carcinogens. Without effective treatment, iron levels progressively increases with deposition in the liver, heart, pancreas, and other endocrine organs. Iron accumulation can result in (i) liver disease that may progress to cirrhosis and hepatocellular carcinoma, (ii) diabetes related both to iron-induced decreases in pancreatic β-cell secretion and increases in hepatic insulin resistance and (iii) heart disease, the leading cause of death in β-thalassemia major and other anemia associated with transfusional iron overload.

Other metals, especially those ions with little or no endogenous function, may find their way into the body and effect damage. Heavy metal ions such as Hg²⁺ can replace ions such as Zn²⁺ in metalloproteins and render them inactive, resulting in serious acute or chronic toxicity that can end in death or cause birth defects. Even more significantly, radioactive isotopes of the lanthanide and actinide series can visit grave illness on an individual exposed to them by mouth, air, or skin contact. Such exposure could result not only from the detonation of a nuclear bomb or a “dirty bomb” composed of nuclear waste, but also from the destruction of a nuclear power facility.

Traditional standard therapies for metal overload include the use of metal chelators such as deferoxamine (DFO, N′-[5-(acetyl-hydroxy-amino)pentyl]-N-[5-[3-(5-aminopentyl-hydroxy-carbamoyl)propanoylamino]pentyl]-N-hydroxy-butane diamide). DFO is an effective metal chelator; unfortunately, it is not orally bioavailable and has a very short half-life in serum. More recently, other metal chelators have been developed for clinical use, but have serious side effects including life-threatening agranulocytosis (deferiprone, Ferriprox), renal and liver toxicity (deferesirox, Exjade). Others are not as effective and require repeated daily doses.

Therefore, there is still a great need for a safe, effective and orally active metal chelator for the treatment of metal overload.

### SUMMARY OF THE INVENTION

It has now been found that certain novel polymorphs of the present invention, and compositions thereof, are useful as metal chelators and exhibit desirable characteristics for the same. In general, these polymorphs, and pharmaceutically acceptable compositions thereof, are useful for treating or lessening the severity of a variety of diseases or disorders as described in detail herein.

In certain aspects, the invention provides for polymorph form D and polymorph form E of Compound 1:

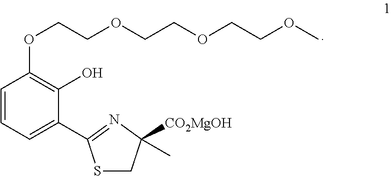

### BRIEF DESCRIPTION OF THE DRAWINGS

FIG. 1. XRPD Patterns of (S)-3′-(OH)-DADFT-PE magnesium salt: FIG. 1a form E from vacuum filtration; FIG. 1b. XRPD Patterns of (S)-3′-(OH)-DADFT-PE magnesium salt: form E from centrifugation.

FIG. 2. XRPD patterns of (S)-3′-(OH)-DADFT-PE Mg under process conditions. From top to bottom: reference Form E pattern, post-filtration (wet), post-centrifugation (wet), post-filtration (dried), and post-centrifugation (dried).

FIG. 3. XRPD Patterns of (S)-3′-(OH)-DADFT-PE magnesium salt: form D from DVS.

### DETAILED DESCRIPTION OF THE INVENTION

### General Description of Certain Aspects of the Invention:

In certain aspects, the invention provides for salts and polymorphs of compounds of Formula I:

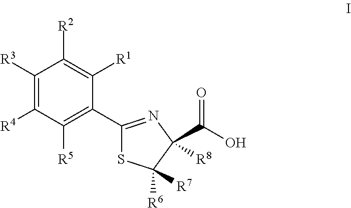

wherein:
R¹, R², R³, R⁴, and R⁵ are independently chosen from hydrogen, hydroxy, alkyl, arylalkyl, alkoxy, and CH₃O((CH₂)ₙ—O)ₘ—, any of which may be optionally substituted;
R⁶, R⁷, and R⁸ are independently chosen from hydrogen, halogen, hydroxy, lower alkyl, and lower alkoxy;
m is an integer from 0 to 8; and
n is an integer from 0 to 8.

In some embodiments, R¹ is OH.

In some embodiments, R² is CH₃O((CH₂)ₙ—O)ₘ—. In some embodiments, R² is CH₃O((CH₂)ₙ—O)ₘ—, n is 2 and m is 3.

In some embodiments, R³ is CH₃O((CH₂)ₙ—O)ₘ—. In some embodiments, R³ is CH₃O((CH₂)ₙ—O)ₘ—, n is 2 and m is 3.

In some embodiments, R² or R³ is CH₃O((CH₂)ₙ—O)ₘ—. In some embodiments, R² or R³ is CH₃O((CH₂)ₙ—O)ₘ—, n is 2 and m is 3.

In some embodiments, the invention provides a salt or polymorph of a compound of Formula I 3′-desazadesferrithiocin polyether.

In certain embodiments, salts of Formula I are solid.

In further embodiments, salts of Formula I are crystalline.

In further embodiments, salts of Formula I are amorphous.

It will be appreciated that where the present disclosure refers to a compound of Formula I, salts and polymorphs of a compound of Formula I are also included.

In some embodiments, compounds disclosed herein are salts or polymorphs thereof having structural Formula II:

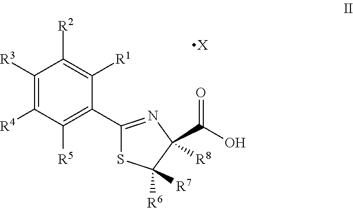

or a salt or polymorph thereof,
wherein:

X is a counterion; and

each of R¹, R², R³, R⁴, R⁵, R⁶, R⁷, and R⁸ are as defined above and described in classes and subclasses herein, both singly and in combination. As used herein, the phrase “X is a counterion” may be inferred and a corresponding charges on each moiety be assumed to be present or absent. For example, if X is a monovalent cation such as Mg(OH)⁺, it may be inferred that the coupled compound has lost a proton to form an ionic bond with X, despite the formulae being drawn to explicitly show all protons in place. Similarly, when X is an anion, the coupled compound takes on cationic character. As used herein, the term counterion encompasses all possible placement where on a compound a counterion has bound and ratios of charges. Additionally, counterions and compounds may combine in uneven molar ratios to form solid salts. As those of skill in the art will recognize, different ratios of counterions may form stable arrangements and solid forms, including 1:1, 2:1, and 3:1 based on preferred oxidation states of each ion, salt formation conditions (including solvent), etc. All such forms are contemplated here.

In certain embodiments, R⁸ is chosen from hydrogen and methyl.

In further embodiments, R⁶ and R⁷ are independently chosen from hydrogen and methoxy.

In further embodiments, R¹ is hydroxy.

In further embodiments, R², R³, R⁴, and R⁵ are independently chosen from hydrogen and CH₃O((CH₂)ₙ—O)ₘ—.

In certain embodiments, salts of Formula II are solid.

In further embodiments, salts of Formula II are crystalline.

In certain embodiments, the counterion X of Formula II is chosen from lysine, N-methyl-D-glucamine (NMG), tromethamine, calcium, magnesium, potassium, di-potassium, sodium, di-sodium, zinc, and piperazine. In some embodiments, X includes one or more metal cations and optionally, as required by charge, an anion such as halide, carbonate, bicarbonate, hydroxide, carboxylate, sulfate, bisulfate, phosphate, nitrate, alkoxy having from 1 to 6 carbon atoms, sulfonate, and aryl sulfonate (e.g., MgOH⁺).

In further embodiments, salts and polymorphs thereof have structural formula III:

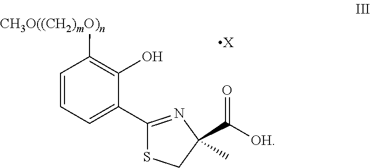

In further embodiments, salts and polymorphs thereof have structural formula IIIa:

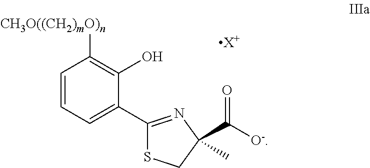

In certain embodiments, the salts and polymorphs thereof have structural formula Mb:

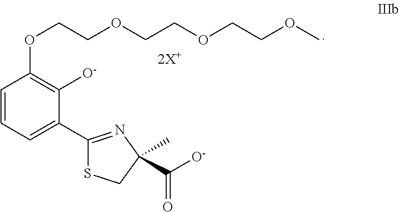

In certain embodiments, salts of Formula III, IIIa, and IIIb are solid.

In further embodiments, salts of Formula III, IIIa, and IIIb are crystalline.

In further embodiments, salts of Formula III, IIIa, and IIIb are amorphous.

In further embodiments, the counterion X is chosen from calcium, magnesium, potassium, di-potassium, sodium, di-sodium, zinc, and piperazine, and optionally as required by charge, includes an anion (e.g., MgOH⁺). Exemplary such anions include, without limitation, halide, carbonate, bicarbonate, hydroxide, carboxylate, sulfate, bisulfate, phosphate, nitrate, alkoxy having from 1 to 6 carbon atoms, sulfonate, and aryl sulfonate.

In further embodiments, m is 2 and n is 3.

In further embodiments, the salt is the magnesium salt, or a polymorph thereof.

In further embodiments, the salt is magnesium 3′-desazadesferrithiocin polyether hydroxide or a polymorph thereof.

In further embodiments, said polymorph of magnesium 3′-desazadesferrithiocin polyether hydroxide is Form A.

In certain embodiments, salts and polymorphs thereof have structural formula IV:

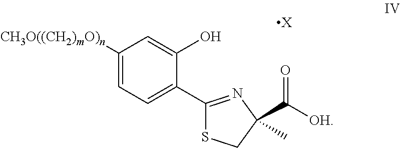

In further embodiments, salts and polymorphs thereof have structural formula IVa:

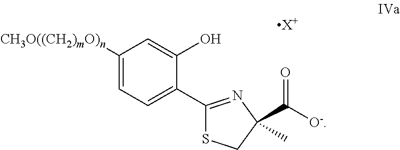

In certain embodiments, salts of Formula IV and IVa are solid.

In further embodiments, salts of Formula IV and IVa are crystalline.

In further embodiments, salts of Formula IV and IVa are amorphous.

In further embodiments, X is chosen from lysine, NMG, tromethamine, calcium, and magnesium, and optionally as required by charge includes an anion such as halide, carbonate, bicarbonate, hydroxide, carboxylate, sulfate, bisulfate, phosphate, nitrate, alkoxy having from 1 to 6 carbon atoms, sulfonate, and aryl sulfonate (e.g., MgOH⁺).

In certain embodiments, salts and polymorphs thereof have structural formula V:

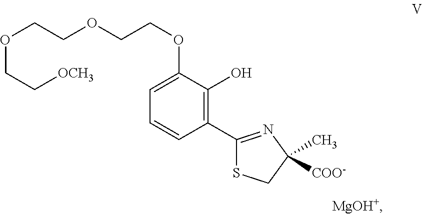

or, equivalently, magnesium hydroxide (S)-3′-desazadesferrithiocin polyether (Mg(OH).3′-DADFT-PE), or (S)-2-(2-hydroxy-3-(2-(2-(2-methoxyethoxyl)ethoxy)ethoxy)phenyl)-4-methyl-4,5-dihydrothiazole-4-carboxylate magnesium hydroxide.

In certain embodiments, salts and polymorphs thereof have structural formula Va:

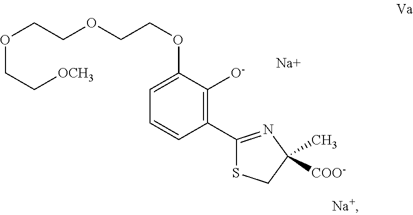

or, equivalently, di-sodium (S)-3′-desazadesferrithiocin polyether (Mg(OH).3′-DADFT-PE), or (S)-2-(2-hydroxy-3-(2-(2-(2-methoxyethoxyl)ethoxy)ethoxy)phenyl)-4-methyl-4,5-dihydrothiazole-4-carboxylate disodium.

In certain embodiments, salts of Formula V and Va are solid.

In further embodiments, salts of Formula V and Va are crystalline.

In certain embodiments, a suitable salt is selected from the group consisting of calcium, magnesium, potassium, di-potassium, sodium, di-sodium, zinc, piperazine, and combination thereof, and optionally as required by charge, includes an anion such as halide, carbonate, bicarbonate, hydroxide, carboxylate, sulfate, bisulfate, phosphate, nitrate, alkoxy having from 1 to 6 carbon atoms, sulfonate, and aryl sulfonate (e.g., MgOH⁺).

In certain embodiments, a suitable salt is (S)-4,5-Dihydro-2-[2-hydroxy-3-(3,6,9-trioxadecyloxyl)phenyl]-4-methyl-4-thiazolecarboxylate magnesium hydroxide. In other embodiments, a suitable salt according to the present invention is (S)-4,5-Dihydro-2-[2-hydroxy-3-(3,6,9-trioxadecyloxyl)phenyl]-4-methyl-4-thiazolecarboxylate magnesium hydroxide Form D polymorph. In other embodiments, a suitable salt according to the present invention is (S)-4,5-Dihydro-2-[2-hydroxy-3-(3,6,9-trioxadecyloxyl)phenyl]-4-methyl-4-thiazolecarboxylate magnesium hydroxide Form E polymorph.

In some embodiments, a suitable salt is a 3′-desazadesferfithiocin polyether di-sodium salt.

In certain embodiments, the invention provides a solid form of Compound 1:

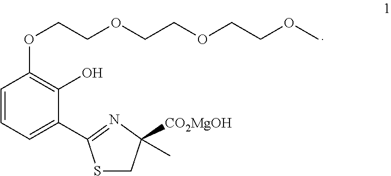

In certain embodiments, the solid form is crystalline.

In various embodiments, the solid form is Form D.

In various embodiments, the solid form is Form E.

Polymorphs of the invention include form D and form E of Compound 1:

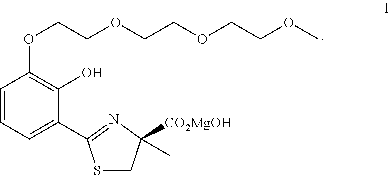

It would be desirable to provide a solid form of Compound 1 that, as compared to Compound 1, imparts characteristics such as improved aqueous solubility, stability and ease of formulation. Accordingly, the present invention provides several solid forms of Compound 1.

Exemplary solid forms are described in more detail below.

In other embodiments, the present invention provides Compound 1 substantially free of impurities. As used herein, the term “substantially free of impurities” means that the compound contains no significant amount of extraneous matter. Such extraneous matter may include starting materials, residual solvents, or any other impurities that may result from the preparation of, and/or isolation of, Compound 1. In certain embodiments, at least about 80% by weight of Compound 1 is present. In certain embodiments, at least about 85% by weight of Compound 1 is present. In certain embodiments, at least about 90% by weight of Compound 1 is present. In certain embodiments, at least about 95% by weight of Compound 1 is present. In certain embodiments, at least about 93% by weight of Compound 1 is present. In still other embodiments of the invention, at least about 99% by weight of Compound 1 is present. In certain embodiments, the extraneous matter is water.

According to one embodiment, Compound 1 is present in an amount of at least about 80, 85, 90, 92, 93, 94, 95, 96, 97, 97.5, 98.0, 98.5, 99, 99.5, or 99.8 weight percent where the percentages are based on the total weight of the composition. According to another embodiment, Compound 1 contains no more than about 3.0 area percent HPLC of total organic impurities and, in certain embodiments, no more than about 1.5 area percent HPLC total organic impurities relative to the total area of the HPLC chromatogram. In other embodiments, Compound 1 contains no more than about 8.0 area percent HPLC of any single impurity; no more than about 7.0 area percent HPLC of any single impurity, no more than about 5.0 area percent HPLC of any single impurity, no more than about 2.0 area percent HPLC of any single impurity, no more than about 1.0 area percent HPLC of any single impurity, no more than about 0.6 area percent HPLC of any single impurity, and, in certain embodiments, no more than about 0.5 area percent HPLC of any single impurity, relative to the total area of the HPLC chromatogram.

The structure depicted for Compound 1 is also meant to include all tautomeric forms of Compound 1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

Solid Forms of Compound 1:

It has been found that Compound 1 can exist in a variety of solid forms. Such forms include polymorphs and amorphous forms. The solid forms can be solvates, hydrates and unsolvated forms of Compound 1. All such forms are contemplated by the present invention. In certain embodiments, the present invention provides Compound 1 as a mixture of one or more solid forms of Compound 1.

As used herein, the term “polymorph” refers to the different crystal structures (of solvated or unsolvated forms) in which a compound can crystallize.

As used herein, the term “solvate” refers to a solid form with either a stoichiometric or non-stoichiometric amount of solvent (e.g., a channel solvate). For polymorphs, the solvent is incorporated into the crystal structure. Similarly, the term “hydrate” refers to a solid form with either a stoichiometric or non-stoichiometric amount of water. For polymorphs, the water is incorporated into the crystal structure.

As used herein, the term “about”, when used in reference to a degree 2-theta value refers to the stated value ±0.3 degree 2-theta. In certain embodiments, “about” refers to ±0.2 degree 2-theta or ±0.1 degree 2-theta.

In certain embodiments, Compound 1 is a crystalline solid. In other embodiments, Compound 1 is a crystalline solid substantially free of amorphous Compound 1. As used herein, the term “substantially free of amorphous Compound 1” means that the compound contains no significant amount of amorphous Compound 1. In certain embodiments, at least about 95% by weight of crystalline Compound 1 is present. In still other embodiments of the invention, at least about 97%, 98% or 99% by weight of crystalline compound 1 is present.

In certain embodiments, Compound 1 is a mesophase (liquid crystal).

In certain embodiments, Compound 1 is an amorphous material.

In certain embodiments, Compound 1 is a solvated crystal.

In certain embodiments, Compound 1 is neat or unsolvated crystal form and thus does not have any water or solvent incorporated into the crystal structure.

In certain embodiments, Compound 1 can exist in neat (i.e., anhydrous) crystal forms, or polymorphs.

In some embodiments, the present invention provides a polymorphic form of Compound 1 referred to herein as Form D. In other embodiments, the present invention provides a polymorphic form of Compound 1 referred to herein as Form E.

In certain embodiments, the present invention provides Form D of Compound 1. According to one embodiment, Form D of Compound 1 is characterized in that it has one or more peaks in its powder X-ray diffraction pattern selected from those at about 3.1, about 6.4, about 6.9, and about 18.3 degrees 2-theta. In some embodiments, Form D of Compound 1 is characterized in that it has two or more peaks in its powder X-ray diffraction pattern selected from those at about 3.1, about 6.4, about 6.9, and about 18.3 degrees 2-theta. In certain embodiments, Form D of Compound 1 is characterized in that it has three or more peaks in its powder X-ray diffraction pattern selected from those at about 3.1, about 6.4, about 6.9, and about 18.3 degrees 2-theta. In particular embodiments, Form D of Compound 1 is characterized in having substantially all of the peaks in its X-ray powder diffraction pattern selected from those at about 3.1, about 6.4, about 6.9, and about 18.3 degrees 2-theta.

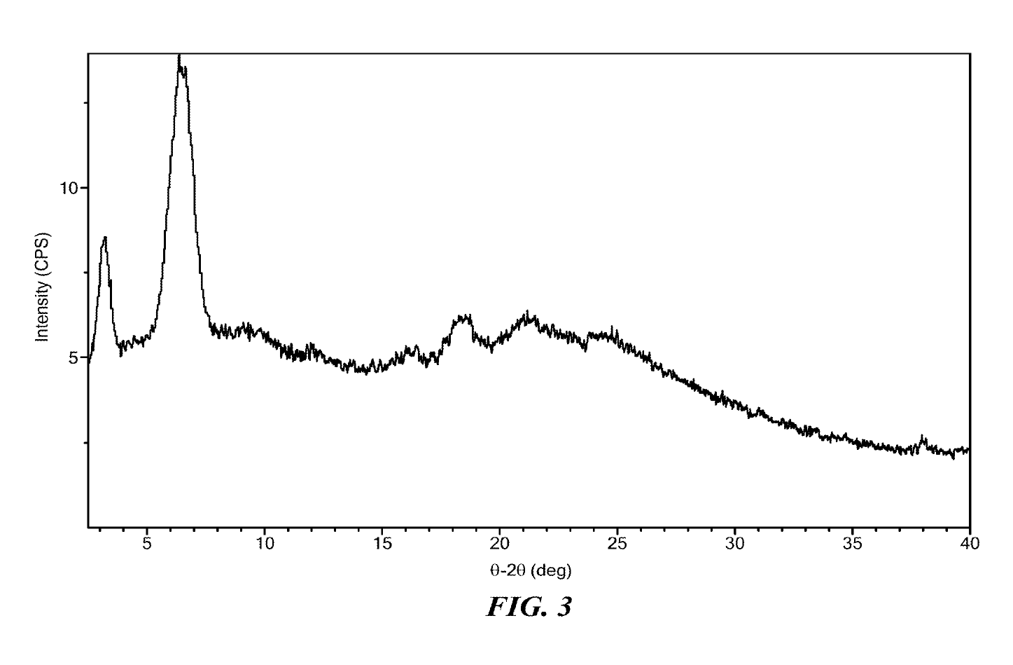

According to one aspect, Form D of Compound 1 has a powder X-ray diffraction pattern substantially similar to that depicted in FIG. 3.

In certain embodiments, the present invention provides Form E of Compound 1. According to another embodiment, Form E of Compound 1 is characterized in that it has one or more peaks in its powder X-ray diffraction pattern selected from those at about 4.1, about 6.0, about 6.1, about 6.9, about 7.5 and about 8.9 degrees 2-theta. In some embodiments, Form E of Compound 1 is characterized in that it has two or more peaks in its powder X-ray diffraction pattern selected from those at about 4.1, about 6.0, about 6.1, about 6.9, about 7.5 and about 8.9 degrees 2-theta. In certain embodiments, Form E of Compound 1 is characterized in that it has three or more peaks in its powder X-ray diffraction pattern selected from those at about 4.1, about 6.0, about 6.1, about 6.9, about 7.5 and about 8.9 degrees 2-theta. In particular embodiments, Form E of Compound 1 is characterized in having substantially all of the peaks in its X-ray powder diffraction pattern selected from those at about 4.1, about 6.0, about 6.1, about 6.9, about 7.5 and about 8.9 degrees 2-theta.

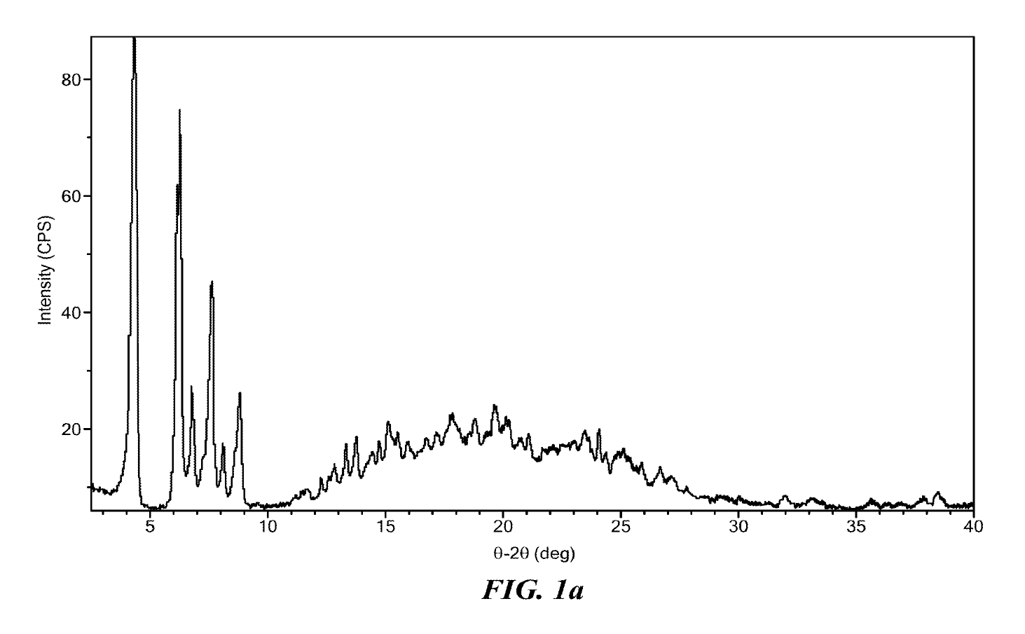

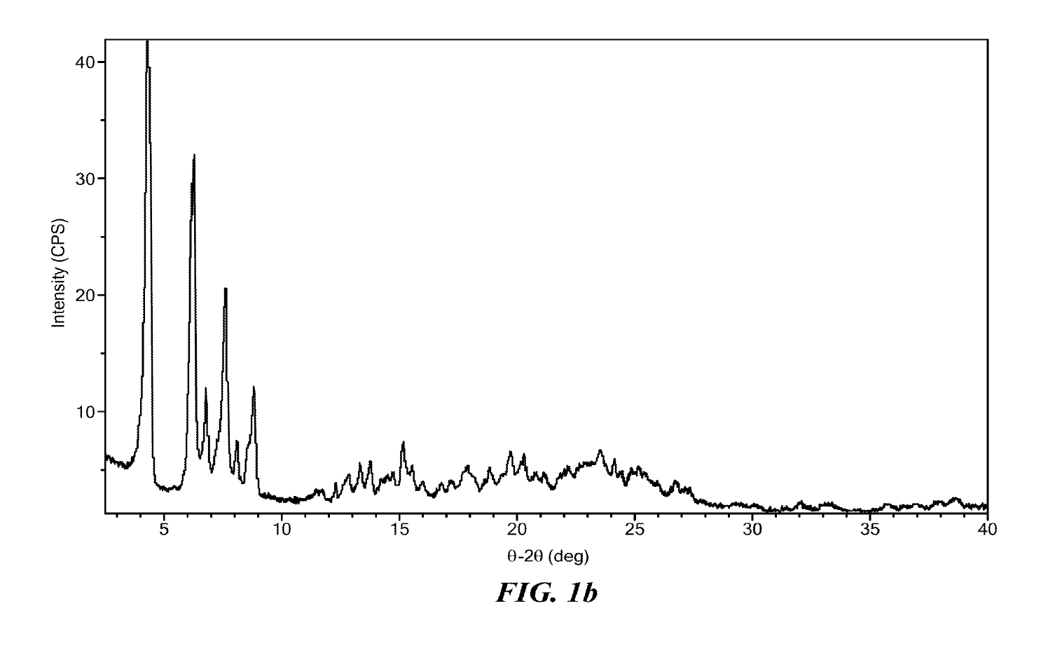

According to one aspect, Form E of Compound 1 has a powder X-ray diffraction pattern substantially similar to that depicted in FIG. 1.

According to another embodiment, the present invention provides compound 1 as an amorphous solid. Amorphous solids are well known to one of ordinary skill in the art and are typically prepared by such methods as lyophilization, melting, and precipitation from supercritical fluid, among others.

### DEFINITIONS

As used herein, when ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The terms “benzo” and “benz,” as used herein, alone or in combination, refer to the divalent group C₆H₄=derived from benzene. Examples include benzothiophene and benzimidazole.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “hydroxy,” as used herein, alone or in combination, refers to —OH.

The term “lower,” as used herein, alone or in a combination, where not otherwise specifically defined, means containing from 1 to and including 6 carbon atoms.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridine, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The compounds disclosed herein can exist as therapeutically acceptable salts. Such salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid.

In addition to specific exemplary salts described above, representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, zinc, and calcium salts of the compounds disclosed herein, and the like.

Basic addition salts can be prepared during the final isolation and purification of the compounds, often by reacting a carboxy group with a suitable base such as the hydroxide, carbonate, or bicarbonate of a metal cation or with ammonia or an organic primary, secondary, or tertiary amine. The cations of therapeutically acceptable salts include, without limitation, lithium, sodium (e.g., NaOH), potassium (e.g., KOH), calcium (including Ca(OH)₂), magnesium (including Mg(OH)₂ and magnesium acetate), zinc, (including Zn(OH)₂ and zinc acetate) and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-diethylethanolamine, N,N′-dimethylethanolamine, triethanolamine, and tromethamine. Basic amino acids such as 1-glycine and 1-arginine, and amino acids which may be zwitterionic at neutral pH, such as betaine (N,N,N-trimethylglycine) are also contemplated. See, for example, Berge et al. (1977) “Pharmaceutical Salts”, J. Pharm. Sci. 66:1-19; incorporated herein by reference.

In certain embodiments, the salts may include lysine, N-methyl glutarate (NMG), tromethamine, calcium, magnesium, potassium, di-potassium, sodium, di-sodium, zinc, and piperazine salts of compounds disclosed herein. In some embodiments, the salts include one or more metal cations and, as required by charge, an anion such as halide, carbonate, bicarbonate, hydroxide, carboxylate, sulfate, bisulfate, phosphate, nitrate, alkoxy having from 1 to 6 carbon atoms, sulfonate, and aryl sulfonate (e.g., MgOH⁺).

Salts disclosed herein may combine in 1:1 molar ratios, and in fact this is often how they are initially synthesized. However, it will be recognized by one of skill in the art that the stoichiometry of one ion in a salt to the other may be otherwise. Salts shown herein may be, for the sake of convenience in notation, shown in a 1:1 ratio; all possible stoichiometric arrangements are encompassed by the scope of the present invention.

The terms, “polymorphs” and “polymorphic forms” and related terms herein refer to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

Techniques for characterizing polymorphs include, but are not limited to, differential scanning calorimetry (DSC), X-ray powder diffractometry (XRPD), thermal gravimetric analysis (TGA), dynamic vapor sorption/desorption (DVS), single crystal X-ray diffractometry, vibrational spectroscopy, e.g. IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

Amorphous form: As used herein, the term “amorphous form” refers to a noncrystalline form of a substance.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a hamster, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bioavailability: As used herein, the term “bioavailability” generally refers to the percentage of the administered dose that reaches the blood stream of a subject.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Chelation: As used herein, the term “chelation” means to coordinate (as in a metal ion) with and inactivate. Chelation also includes decorporation, a term which itself encompasses chelation and excretion.

Compound: As used herein, the term “compound” is meant to be interchangeable with the term “active compound” or “drug,” and refers to a compound having beneficial prophylactic and/or therapeutic properties when administered to a patient and/or activity against a biological target which is associated with a disease.

Counterion: When the phrase “X is a counterion” is used in any formulae herein, and neither the compound nor the counterion is drawn showing explicit ionic character, such ionic character may be inferred and a corresponding charges on each moiety be assumed to be present or absent. For example, if X is a monovalent cation such as Mg(OH)⁺, it may be inferred that the coupled compound has lost a proton to form an ionic bond with X, despite the formulae being drawn to explicitly show all protons in place. Similarly, when X is an anion, the coupled compound takes on cationic character. As used herein, the term counterion encompasses all possible placement where on a compound a counterion has bound and ratios of charges. Additionally, counterions and compounds may combine in uneven molar ratios to form solid salts. As those of skill in the art will recognize, different ratios of counterions may form stable arrangements and solid forms, including 1:1, 2:1, and 3:1 based on preferred oxidation states of each ion, salt formation conditions (including solvent), etc. All such forms are contemplated here.

Desolvated solvate: The term, “desolvated solvate,” as used herein, refers to a crystal form of a substance which can only be made by removing the solvent from a solvate.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment. The “dosage strength” refers to the total drug content of the dosage form.

Excipient: As used herein, the term “excipient” refers to any inert substance added to a drug and/or formulation for the purposes of improving its physical qualities (i.e. consistency), pharmacokinetic properties (i.e. bioavailability), pharmacodynamic properties and combinations thereof.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease as the individual being treated, who is about the same age as the individual being treated (to ensure that the stages of the disease in the treated individual and the control individual(s) are comparable).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Pharmaceutically acceptable: As used herein, the term “pharmaceutically-acceptable” refers to any entity or composition that does not produce an undesirable allergic or antigenic response when administered to a subject.

Prodrug: As used herein, the term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

Solid: As used herein, “solid” when referring to a salt form means relatively solid, at room temperature, and/or containing a substantial amount of solids. A solid may be amorphous in form and/or be a solvated solid with some quantity of residual or coordinated of solvent molecules. A crystalline salt is an example of a solid. By way of example, a wax could be considered a solid, whereas an oil would not be. A “solid composition” as used herein includes a salt of a compound, or a polymorph or amorphous solid form thereof.

Solvate: As used herein, the term “solvate” refers to a crystal form of a substance which contains solvent. The term “hydrate” refers to a solvate wherein the solvent is water.

Stability: As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith.

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### General Methods of Providing Compound 1:

Certain compounds, salts, and polymorphs from which pharmaceutical compositions as disclosed herein may be formed can be synthesized as described in US 20100137383 and Bergeron, R J et al., “Design, Synthesis, and Testing of Non-Nephrotoxic Desazadesferrithiocin Polyether Analogues,” J Med Chem. 2008, 51(13), 3913-23, which are hereby incorporated by reference in their entireties. Additional synthetic protocols for compounds disclosed herein may be found in US20080214630A1 published Sep. 4, 2008; US20100093812A1, published Apr. 15, 2010, and W02011017054A2, published Feb. 10, 2011.

Compound 1 is prepared according to the methods described in detail in the WO2010/009120 (PCT/US2009/050532) application, the entirety of which is hereby incorporated herein by reference. The various solid forms of Compound 1 can be prepared by dissolving compound 1 in various suitable solvents and then causing Compound 1 to return to the solid phase. Specific combinations of solvents and conditions under which Compound 1 return to the solid phase are discussed in greater detail in the Examples.

A suitable solvent may solubilize Compound 1, either partially or completely. Examples of suitable solvents useful in the present invention are a protic solvent, a polar aprotic solvent, or mixtures thereof. In certain embodiments, suitable solvents include an ether, an ester, an alcohol, a ketone, or a mixture thereof. In certain embodiments, the suitable solvent is methanol, ethanol, isopropanol, or acetone wherein said solvent is anhydrous or in combination with water, methyl tert-butyl ether (MTBE) or heptane. In other embodiments, suitable solvents include tetrahydrofuran, 1,4-dioxane, dimethylformamide, dimethylsulfoxide, glyme, diglyme, methyl ethyl ketone, N-methyl-2-pyrrolidone, methyl t-butyl ether, t-butanol, n-butanol, and acetonitrile. In another embodiment, the suitable solvent is anhydrous ethanol. In some embodiments, the suitable solvent is MTBE.

According to another embodiment, the present invention provides a method for preparing a solid form of Compound 1, comprising the steps of dissolving Compound 1 with a suitable solvent and optionally heating to form a solution thereof; and isolating Compound 1.

As described generally above, Compound 1 is dissolved in a suitable solvent, optionally with heating. In certain embodiments, Compound 1 is dissolved at about 50 to about 60° C. In other embodiments, Compound 1 is dissolved at about 50 to about 55° C. In still other embodiments, Compound 1 is dissolved at the boiling temperature of the solvent. In other embodiments, Compound 1 is dissolved without heating (e.g., at ambient temperature, approximately 20-25° C.).

In certain embodiments, Compound 1 precipitates from the mixture. In another embodiment, Compound 1 crystallizes from the mixture. In other embodiments, Compound 1 crystallizes from solution following seeding of the solution (i.e., adding crystals of Compound 1 to the solution).

Crystalline Compound 1 can precipitate out of the reaction mixture, or be generated by removal of part or all of the solvent through methods such as evaporation, distillation, filtration (e.g., nanofiltration, ultrafiltration), reverse osmosis, absorption and reaction, by adding an anti-solvent (e.g., water, MTBE and/or heptane), by cooling (e.g., crash cooling) or by different combinations of these methods.

As described generally above, Compound 1 is optionally isolated. It will be appreciated that Compound 1 may be isolated by any suitable physical means known to one of ordinary skill in the art. In certain embodiments, precipitated solid Compound 1 is separated from the supernatant by filtration. In other embodiments, precipitated solid Compound 1 is separated from the supernatant by decanting the supernatant.

In certain embodiments, precipitated solid Compound 1 is separated from the supernatant by filtration.

In certain embodiments, isolated Compound 1 is dried in air. In other embodiments isolated Compound 1 is dried under reduced pressure, optionally at elevated temperature.

In some embodiments, isolated Compound 1 is lyophilized. In some embodiments, isolated Compound 1 is spray-dried.

### Uses of Compounds

Inventive polymorphs described herein may be used to effectively treat metal overload. As used herein, the term “metal overload” refers to a condition in which the body has reached its limit to absorb and excrete a particular metal, resulting in an excess amount of the metal accumulated in various tissues inside the body that lead to toxicity or other pathological conditions. Inventive polymorphs described herein may be used to chelate, sequester, reduce, or eliminate such accumulated metals including, but not limited to, iron, heavy metals (e.g., Hg²⁺), uranium, and other radioactive isotopes such as lanthanide and actinide series. As used herein, the term “treat,” “treatment,” or “treating” refers to any method used to reduce metal levels (e.g., iron levels) as compared to a baseline control level and/or partially or completely alleviate, ameliorate, relieve, inhibit, prevent, delay onset of, reduce severity of and/or reduce incidence of one or more symptoms or features of a disease, disorder, and/or condition associated with metal overload.

In some embodiments, the metal overload that can be treated using a method of the invention is uranium overload caused by, for example, radiation poisoning.

In some embodiments, the metal overload that can be treated using a method of the invention is iron overload. In various embodiments, a method of the invention can be used to treat iron overload due to repeated blood transfusion (i.e., transfusional iron overload) or increased iron absorption.

In some embodiments, the present invention is used to treat iron overload. As used herein, the term “iron overload” refers to a condition in which an excess amount of iron accumulate inside a body that leads to toxic or other pathological conditions. Without wishing to be bound by theory, iron overload can be toxic in part through the generation by iron of reactive oxygen species such as H₂O₂. In the presence of Fe²⁺, H₂O₂ is reduced to the hydroxyl radical (HO), a highly reactive species, a process known as the Fenton reaction. The hydroxyl radical reacts very quickly with a variety of cellular constituents and can initiate free radicals and radical-mediated chain processes that damage DNA and membranes, as well as produce carcinogens.

In various embodiments, a use of the invention can be used to treat a subject suffering from anemia that results in increased accumulation of iron in the body either due to need for repeated blood transfusions or increased iron absorption. Exemplary causes of anemia include, but are not limited to, Beta thalassemia major or intermedia, and other anemias including but not limited to non-transfusion dependent Thalassaemia (NTDT—i.e. patients with clinically milder forms of thalassemia, such as β-thalassemia intermedia, α-thalassemia (HbH disease), and HbE/β-thalassemia, who require occasional or no blood transfusions), Blackfan-Diamond anemia, Fanconi's anemia and other inherited bone marrow failure syndromes, Sideroblastic anemia, congenital dyserythropoietic anemias, sickle cell disease, pyruvate kinase deficiency (and other red cell enzyme deficiency causing hemolytic anemia), aplastic anemia, refractory anemias, red cell aplasia, Myelodysplasia (MDS), chronic myelofibrosis, paroxysmal nocturnal hemoglobinuria); from increased absorption of dietary iron (in conditions such as hereditary hemochromatosis and porphyria cutanea tarda); from mal-distribution or redistribution of iron in the body (e.g., resulted from conditions such as atransferrinemia, aceruloplasminemia, and Friedreich's ataxia); from transfusional iron overload from off-therapy leukemias, before and after bone marrow transplant and myelodysplastic syndrome; from diabetes or obesity; and/or from liver diseases (e.g., hepatitis).

In some embodiments, a use of the invention can be used to treat a subject suffering from β-thalassemia-intermedia. In some embodiments, a use of the invention can be used to treat a subject suffering from β-thalassemia-major.

In some embodiments, a use of the invention can be used to treat a subject suffering from iron overload due to repeated blood transfusions as a consequence of the subject suffering from sickle cell disease. In some embodiments, a use of the invention can be used to treat a subject suffering from Myelodysplastic Syndrome (MDS).

Typically, under normal conditions, iron absorption and loss are balanced at about 1 mg/day. Iron overload can be caused by repeated blood transfusion (i.e., transfusional iron overload) or increased iron absorption required in patients suffering from various congenital and acquired anemias. Exemplary causes of anaemia include, but are not limited to, β-thalassemia-major, non-transfusion dependent Thalassaemia (NTDT) such as β-thalassemia-intermedia, Blackfan-Diamond anemia, Sideroblastic anemia, sickle cell disease, aplastic anemia, red cell aplasia, Myelodysplasia (MDS), chronic myelofibrosis, paroxysmal nocturnal hemoglobinuria.

Typically, transfused blood contains 200-250 mg of iron per unit. Hence, patients with β-thalassemia major (TM) or other refractory anemias receiving 2-4 units of blood per month have an annual intake of 5000-10,000 mg of iron or 0.3-0.6 mg/kg per day.

Thus, in some embodiments, iron overload refers to a condition under which a subject has an iron intake greater than 1 mg/day, 5 mg/day, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day or 100 mg/day. In some embodiments, iron overload refers to a condition under which a subject has an iron intake greater than 0.1 mg/kg per day, 0.2 mg/kg per day, 0.3 mg/kg per day, 0.4 mg/kg per day, 0.5 mg/kg per day, 0.6 mg/kg per day, 0.7 mg/kg per day, 0.8 mg/kg per day, or more. In some embodiments, iron overload refers to a condition under which a subject has an iron intake of approximately 0.1 to 0.7 mg/kg per day. In some embodiments, iron overload refers to a condition under which a subject has an iron intake of approximately 0.2 to 0.6 mg/kg per day.

In some embodiments, iron overload refers to a condition under which a subject has an iron intake of approximately 0.3 to 0.5 mg/kg per day. In certain embodiments, iron overload refers to a condition under which a subject has an iron intake of approximately 0.35 to 0.45 mg/kg per day. In certain embodiments, 0.35 mg/kg per day; 0.38 mg/kg per day; 0.43 mg/kg per day; or 0.45 mg/kg per day. In certain embodiments, iron overload refers to a condition under which a subject has an iron intake of approximately 0.38 to 0.64 mg/kg per day. In certain embodiments, 0.38 mg/kg per day; 0.42 mg/kg per day; 0.47 mg/kg per day; or 0.64 mg/kg per day. In certain embodiments, iron overload refers to a condition under which a subject has an iron intake of approximately 0.21 to 0.31 mg/kg per day. In certain embodiments, 0.21 mg/kg per day; 0.25 mg/kg per day; or 0.31 mg/kg per day. In certain embodiments, iron overload refers to a condition under which a subject has an iron intake of approximately 0.24 to 0.28 mg/kg per day. In certain embodiments, 0.24 mg/kg per day; 0.26 mg/kg per day; or 0.28 mg/kg per day. In certain embodiments, iron overload refers to a condition under which a subject has an iron intake of approximately 0.43 to 0.56 mg/kg per day. In certain embodiments, 0.43 mg/kg per day; 0.49 mg/kg per day; or 0.56 mg/kg per day. In certain embodiments, iron overload refers to a condition under which a subject has an iron intake of approximately 0.41 to 0.42 mg/kg per day.

Without effective treatment, iron overload may cause iron levels to progressively increase with deposition in various tissues including, but not limited to, the liver, heart, pancreas, and other endocrine organs. Iron accumulation may also produce (i) liver disease that may progress to cirrhosis and hepatocellular carcinoma, (ii) diabetes related both to iron-induced decreases in pancreatic β-cell secretion and increases in hepatic insulin resistance and (iii) heart disease.

Polymorphs according to the present invention may be used to treat various iron overload conditions including, but not limited to, iron overload resulted from red blood cells chronic transfusion (necessary in conditions such as beta thalassemia major or intermedia, and other anemias including but not limited to non-transfusion dependent Thalassaemia (NTDT—i.e. patients with clinically milder forms of thalassemia, such as β-thalassemia intermedia, α-thalassemia (HbH disease), and HbE/β-thalassemia, who require occasional or no blood transfusions), Blackfan-Diamond anemia, Fanconi's anemia and other inherited bone marrow failure syndromes, Sideroblastic anemia, congenital dyserythropoietic anemias, sickle cell disease, pyruvate kinase deficiency (and other red cell enzyme deficiency causing hemolytic anemia), aplastic anemia, refractory anemias, red cell aplasia, Myelodysplasia (MDS), chronic myelofibrosis, paroxysmal nocturnal hemoglobinuria); from increased absorption of dietary iron (in conditions such as hereditary hemochromatosis and porphyria cutanea tarda); from mal-distribution or redistribution of iron in the body (e.g., resulted from conditions such as atransferrinemia, aceruloplasminemia, and Fredreich's ataxia); from transfusional iron overload from off-therapy leukemias, before and after bone marrow transplant and myelodysplastic syndrome; from diabetes or obesity; and/or from liver diseases (e.g., hepatitis).

In various embodiments, polymorphs of the present invention may be used to treat acute iron toxicity from ingestion or infusion of iron; to reduce total body iron secondary to transfusion or excess iron dietary absorption; and/or for maintenance of iron balance after total body iron has been satisfactorily reduced and only excess daily transfusional or dietary iron needs to be excreted. Thus, in some embodiments, administration of a polymorph described herein results in excretion between 0.2 and 0.5 mg Fe/kg body weight of the patient per day (e.g., about 0.2, 0.3, 0.4, or 0.5 mg Fe/kg body weight of the patient per day). In some embodiments, this amount of excretion is recommended for chronic iron overload secondary to transfusion. In some embodiments, administration of a polymorph described herein results in excretion between 0.25-0.5 mg Fe/kg/d of patient body weight (e.g., about 0.25, 0.30, 0.35, 0.40, 0.45, 0.50 mg Fe/kg body weight of the patient per day). In some embodiments, this amount of excretion is recommended to achieve iron balance neutrality and/or for maintenance treatment.

In some embodiments, the efficacy of treatment according to the present invention may be measured by iron-clearing efficiency. As used herein, the term “iron-clearing efficiency (ICE)” refers to the molar efficiency or efficaciousness of a given dose or concentration of chelator in clearing iron from the body or one of its tissues, organs or parts. Efficaciousness in turn concerns quantity of iron removed from a target system (which may be a whole body, an organ, a tissue or other) in a unit of time. Iron clearing efficiency (ICE) is calculated by subtracting total iron excretion before treatment from total iron excreted after treatment and dividing that value by the theoretical amount of iron that could have been bound by the dose of chelator administered times 100.

In some embodiments, measurement of certain markers will be used as a proxy to assess therapeutic efficacy. In iron overload diseases, for example, the free iron species, non-transferrin-bound iron (NTBI), and labile plasma iron (LPI, also called redox-active iron) in the circulation, and the labile and chelatable iron pool within the cells, are responsible for iron toxicity through the generation of reactive oxygen species. The characteristic features of advanced iron overload are dysfunction and failure of vital organs such as liver and heart in addition to endocrine dysfunctions. For the estimation of body iron, there are direct and indirect methods available. See, e.g, Kohgo Y “Body iron metabolism and pathophysiology of iron overload,” Int J Hematol., 2008 88(1): 7-15 (epub 2008 Jul. 2); Angelucci E et al. “Hepatic Iron Concentration and Total Body Iron Stores in Thalassemia Major,” NEJM, 2000 343(5): 327-331.

In some embodiments, measurement of serum ferritin can be used for monitoring efficacy. Ferritin is a globular cytoplasmic protein consisting of 25 heterodimeric subunits of H and L that stores iron as ferric hydroxide phosphate in a controlled manner, which may be found in the plasma in low concentration. By quantitative phlebotomy, it has been demonstrated that serum ferritin (SF) correlates with total body iron stores. However, the level of SF may be affected by acute and chronic inflammation and infections. There is also a difference between the standard values of SF concentration in males and females (normal range 10-220 μg/L in males; 10-85 μg/L in females). Therefore, data should be interpreted carefully when using SF as a biological marker for evaluation of body iron stores. Clinically, in order to detect organ dysfunctions, serum ferritin determinations should be conducted once every 1-3 months. According to the guidelines of the International MDS Symposium, 1,000 μg/L represents the threshold of the target SF value at which iron chelation therapy should be initiated in patients with transfusion iron overload. When serum ferritin levels exceed 1,500 μg/L, patients should be examined for the symptoms of cardiac failure or arrhythmias, and periodical cardiac echograms may also be useful in diagnosis. The concentration of heart iron is increased when SF levels become greater than 1,800 μg/L, and the prevalence of cardiac events is significantly increased when SF levels are more than 2,500 μg/L.

The present disclosure recognizes that even serum ferritin levels greater than 500 μg/L can be cause for iron chelation therapy. Thus, in some embodiments, the present invention may be used to treat a subject that has a serum ferritin level greater than about 500 μg/L (e.g., greater than about 600 μg/L, 700 μg/L, 800 μg/L, 900 μg/L). In some embodiments, the present invention may be used to treat a subject that has a serum ferritin level greater than about 800 μg/L. In some embodiments, the present invention may be used to treat a subject that has a serum ferritin level greater than about 1,000 μg/L (e.g., greater than about 1,200 μg/L, 1,500 μg/L, 1,800 μg/L, 2,000 μg/L, 2,200 μg/L, or 2,500 μg/L). In various embodiments, administration of a polymorph according to the present invention results in reduction of serum ferritin level in the subject as compared to a baseline control. In some embodiments, administration of a polymorph according to the present invention results in the serum ferritin level in the subject being treated below 1,000 μg/L.

An alternate method of assessing iron level in the body is via the measurement of labile plasma iron, a redox active form of non-transferrin bound iron that is chelatable, making it potentially available for transport into extrahepatic tissues. LPI can be accurately and reproducibly assayed by fluorescent method; see, e.g., Esposito B P et al., “Labile plasma iron in iron overload: redox activity and susceptibility to chelation,” Blood, 2003, 102(7):2670-7 (Epub 2003 Jun. 12) and Wood, J C et al., “Relationship between labile plasma iron, liver iron concentration and cardiac response in a deferasirox monotherapy trial,” Haematologica, 2011 96(7): 1055-1058 (epub 2011 March 10). LPI measurements may be influenced by antioxidant and iron-binding activities of sera. Since LPI measurements are performed on intact serum or plasma, they should represent the sum of the pro-oxidant potential of the chelatable iron and the antioxidant activity of the sample. The total antioxidant activity of human plasma/serum has been estimated in the range of 1 mM and can be influenced by a variety of factors including diet and clinical conditions. Therefore, it is possible that sera containing similar concentrations of NTBI might have different levels of LPI, due to masking by antioxidants. It has also been suggested that chronic control of circulating LPI may be an important goal for iron chelation therapy in order to prevent oxidative damage, and to lower the risk of extrahepatic organ dysfunction.

Alternatively, iron concentration in a target organ or tissue may be measured directly. The measurement of liver iron concentration (LIC) by liver biopsy has traditionally been viewed as the most reliable means to assess body iron storage. The LIC level may also be determined by magnetic resonance imaging (MRI). The liver is the most important organ for iron storage with the largest capacity to sequester excess iron. In patients with β-thalassemia, the risk of organ dysfunction is increased when LIC values are greater than 7 mg/g (liver, dry weight), and LIC levels of over 15 mg/g (liver, dry weight) increase the risk of early cardiac death due to iron deposition in the myocardium. Studies in the deferasirox clinical development program in β-thalassemia also demonstrated a correlation between the reduction in LIC and SF values (R=0.63). In some embodiments, the present invention may be used to treat a subject that has an LIC level greater than about 7 mg/g (liver, dry weight) (e.g., greater than about 8, 9, 10, 11, 12, 13, 14, or 15 mg/g (liver, dry weight)). In some embodiments, administration of a polymorph according to the present invention results in reduction of the LIC level in the subject as comparted to a baseline control. In some embodiments, administration of a polymorph according to the present invention results in the LIC level in the subject being treated below 7 mg/g (liver, dry weight).

The determination of cardiac iron concentration is clinically important because one of the major causes of death in iron overload is sudden cardiac arrest. Additionally, pancreatic beta cells are another important target of iron toxicity, which cause glucose intolerance and diabetes mellitus.

Recently, physical detection methods using magnetic resonance imaging (MRI) and superconducting quantum interference devices (SQUID) have become available to indirectly estimate iron concentration in liver, pancreas, and myocardium. In some embodiments, the cardiac iron level may be measured by MRI R2* or T2* MRI. It has been reported that a shortening of myocardial T2* to less than 20 ms (implying increased myocardial iron above normal) is associated with an increased likelihood of decreased left ventricular ejection fraction (LVEF), whereas patients with T2* values greater than 20 ms have a very low likelihood of decreased LVEF. In some embodiments, the present invention may be used to treat a subject that has a myocardial T2* value less than about 20 ms (e.g., less than about 18, 16, 14, 10, 8, 6, 4, 2 ms). In some embodiments, administration of a polymorph according to the present invention results in the reduction of cardiac iron level in the subject as compared to a baseline control. In some embodiments, the administration of a polymorph according to the present invention results in myocardial T2* value great than 20 ms.

Appropriate baseline controls described herein (e.g., for serum ferritin, LIC, and/or cardiac iron level) are indicative of the pre-treatment levels in the corresponding tissues.

The subject (also referred to as “patient” or “individual”) being treated can be a child, adolescent, or adult human. Besides being useful for human treatment, certain compounds and polymorphs disclosed herein may also be useful for veterinary treatment of companion animals, exotic animals and farm animals, including mammals, rodents, and the like. More preferred animals include horses, dogs, and cats.

Combination Therapy

In some embodiments, polymorphs provided herein for treating diseases, disorders or conditions relating to metal toxicity or overload in a human or animal subject in need of such treatment can be used in combination with one or more additional agents that are beneficial for the treatment of such diseases, disorders or conditions and/or can reduce side effects.

In certain instances, it may be appropriate to administer a polymorph described herein in combination with supplements of essential trace minerals required by the body for proper functioning, for example zinc and magnesium, to replace those which will inadvertently be lost to chelation therapy. Or, by way of example only, the therapeutic effectiveness of a polymorph described herein may be enhanced by co-administration of an adjuvant (i.e., by itself the adjuvant may only have minimal therapeutic benefit, but in combination with another therapeutic agent, the overall therapeutic benefit to the patient is enhanced). Or, by way of example only, the benefit of a polymorph described herein may be enhanced with another therapeutic agent (which also includes a therapeutic regimen) that also has therapeutic benefit for treating metal overload. By way of example only, in a treatment for thalassemia involving administration of a polymorph described herein, increased therapeutic benefit may result by also providing the patient with another therapeutic agent for thalassemia, for example deferoxamine. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain polymorphs as disclosed herein with: deferasirox, deferiprone, deferoxamine, DTPA (diethylene triamine pentaacetic acid), EGTA (ethylene glycol tetraacetic acid), EDTA (ethylenediamine tetraacetic acid), DMSA (dimercaptosuccinic acid), DMPS (dimercapto-propane sulfonate), BAL (dimercaprol), BAPTA (aminophenoxyethane-tetraacetic acid), D-penicillamine, and alpha lipoic acid.

In various embodiments, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to weeks.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### General Procedures

### A. Materials

(S)-3′-(OH)-DADFT-PE was supplied from FerroKin BioSciences and used to prepare amorphous and Form A of (S)-3′-(OH)-DADFT-PE magnesium salt. Solvents and other reagents were purchased from commercial suppliers and were either HPLC or ACS grade and used as received.

### B. Experimental Methods

### 1. Approximate Solubility

a. Solvent Addition Method

A weighed sample was treated with aliquots of the test solvent at ambient temperature. Complete dissolution of the test material was determined by visual inspection. Solubility was estimated based on the total solvent used to provide complete dissolution. The actual solubility may be greater than the value calculated because of the use of solvent aliquots that were too large or due to a slow rate of dissolution. The solubility is expressed as “less than” if dissolution did not occur during the experiment. If complete dissolution was achieved as a result of only one aliquot addition, the solubility is expressed as “greater than”.

b. Gravimetric Method

An aqueous solution was prepared by adding aliquots of water with excess solid present. The mixture was then agitated in a sealed vial at room temperature. After several hours, the mixture was centrifuged. The clear solution was added to a tared vial and the total mass was recorded. The vial was then placed under vacuum at slightly elevated temperature until the sample was dry. The gravimetric solubility was calculated based on the mass of water lost and the residue remaining in the vial.

### 2. Polymorph Screen

Both thermodynamic and kinetic crystallization techniques were employed. These techniques are described in more detail below. Once solid samples were harvested from crystallization attempts, they were either examined under a microscope for morphology or observed with the naked eye. Any crystalline shape was noted, but sometimes the solid exhibited unknown morphology, due to small particle size. Solid samples were then analyzed by XRPD.

a. Ambient Solution (AS)

Solutions were prepared as close to saturation as possible in various solvents at ambient. This was accomplished by adding an anti-solvent to the solution until turbidity was observed. The sample was left at ambient. Solids that formed were isolated by either filtration or decantation and allowed to dry prior to analysis.

b. Fast Evaporation (FE)

Solutions were prepared in various solvents and sonicated between aliquot additions to assist in dissolution. Once a mixture reached complete dissolution, as judged by visual observation, the solution was filtered through a 0.2-μm nylon or PVDF filter. The filtered solution was allowed to evaporate at ambient. The solids that formed were isolated and analyzed.

c. Slow Cool (SC)

Saturated solutions were prepared in various solvents at elevated temperatures and filtered through a 0.2-μm nylon filter into a vial. Vials were then left on top of the hot plate and allowed to cool to ambient temperature slowly. The resulting solids were isolated by filtration and dried prior to analysis.

d. Slow Evaporation (SE)

Solutions were prepared in various solvents. The solution was then filtered through a 0.2-μm nylon or PVDF filter. The filtered solution was allowed to evaporate at ambient in a vial covered with aluminum foil perforated with pinholes. The solids that formed were isolated and analyzed.

e. Slurry Experiments

Solutions were prepared by adding enough solids to a given solvent so that excess solids were present. The mixture was then agitated in a sealed vial on a slurry wheel at ambient temperature. Solids were isolated by vacuum filtration.

f. Solvent Grinding

Material was placed into an agate milling rotor with a small agate ball. A small amount of solvent was then added. The sample was then ground at 30 hz on a Retesh type MM220 mixermill for approximately 20 or 40 minutes. The resulting solids were isolated and analyzed.

g. Vapor Diffusion (VD)

Solutions were prepared with various solvents at ambient temperature and filtered through a 0.2-nm nylon or PVDF filter. These solutions were placed into an open container. This container was placed in a sealed chamber containing an anti-solvent. The anti-solvent is miscible with and, typically, more volatile than the solvent. The chamber was left undisturbed at ambient.

### C. Instrumental Techniques

### 1. Coulometric Karl-Fischer Analysis (KF)

Coulometric Karl Fischer (KF) analysis for water determination was performed using a Mettler Toledo DL39 Karl Fischer titrator. The sample was placed in the KF titration vessel containing of Hydranal-Coulomat AD and mixed for 60 seconds to ensure dissolution. The sample was then titrated by means of a generator electrode which produces iodine by electrochemical oxidation: 2 I−=>I₂+2e. The sample size was optimized by performing a scoping experiment. Two replicates were obtained to ensure reproducibility. The value reported is the average of the two replicates.

### 2. Differential Scanning Calorimetry (DSC)

Differential scanning calorimetry acquisition and processing parameters are printed on each thermogram. The sample was placed into an aluminum DSC pan. The sample cell was heated under a nitrogen purge. Indium metal was used as the calibration standard.

### 3. Dynamic Vapor Sorption/Desorption (DVS)

Moisture sorption/desorption data were collected on a VTI SGA-100 Vapor Sorption Analyzer. Sorption and desorption data were collected over a range of 5% to 95% relative humidity (RH) at 10% RH intervals under a nitrogen purge. Samples were not dried prior to analysis. Equilibrium criteria used for analysis were less than 0.0100% weight change in 5 minutes, with a maximum equilibration time of 3 hours if the weight criterion was not met. Data were not corrected for the initial moisture content of the samples. Sodium chloride and polyvinylpyrrolidine were used as calibration standards.

### 4. Hot Stage Microscopy

Hot stage microscopy was performed using a Linkam hot stage (model FTIR 600) mounted on a Leica DM LP microscope. Samples were observed using a 20× objective (obj.) with cross polarizers (CP) and lambda compensator. Samples were placed on a coverslip. A second coverslip was then placed over the sample. Each sample was visually observed as the stage was heated. Images were captured using a SPOT Insight™ color digital camera with SPOT Software v. 4.5.9. The hot stage was calibrated using USP melting point standards.

### 5. Nuclear Magnetic Resonance (NMR)

The solution phase ¹H NMR spectra were collected at SSCI or Spectra Data Services, Inc. Acquisition parameters are printed on each page of data. Spectra were referenced to internal tetramethylsilane at 0.0 ppm.

### 6. Thermogravimetry (TG)

Thermogravimetry acquisition and processing parameters are printed on each thermogram. The furnace was heated under nitrogen. Nickel and Alumel™ were used as the calibration standards.

### 7. X-Ray Powder Diffraction (XRPD)

a. PANalytical X'Pert Pro Diffractometer

The specimen was analyzed using Cu radiation produced using an Optix long fine-focus source. An elliptically graded multilayer mirror was used to focus the Cuκα X-rays of the source through the specimen and onto the detector. The specimen was sandwiched between 3-micron thick films, analyzed in transmission geometry, and rotated to optimize orientation statistics. A beam-stop was used to minimize the background generated by air scattering. Soller slits were used for the incident and diffracted beams to minimize axial divergence. Diffraction patterns were collected using a scanning position-sensitive detector (X'Celerator) located 240 mm from the specimen. The data-acquisition parameters of each diffraction pattern are displayed above the image of each pattern. Prior to the analysis a silicon specimen (NIST standard reference material 640c) was analyzed to verify the position of the silicon Ill peak.

b. Inel XRG-3000 Diffractometer

X-ray powder diffraction acquisition and processing parameters are printed on each pattern found in the data section. The instrument is equipped with a CPS (Curved Position Sensitive) detector with a 2θ range of 120°. The monochromator slit was set at 5 mm by 160 μm. The pattern is displayed from 2.5-40°2θ. Instrument calibration was performed using a silicon reference standard.

c. Bruker D-8 Discover Diffractometer

X-ray powder diffraction acquisition and processing parameters are printed on each pattern found in the data section. XRPD patterns were collected with a Bruker D-8 Discover diffractometer and Bruker's General Area Diffraction Detection System (GADDS, v. 4.1.20). An incident beam of Cu Kα radiation was produced using a fine-focus tube (40 kV, 40 rnA), a Gobel mirror, and a 0.5 mm double-pinhole collimator. The sample was placed in a TQSH holder and secured to a translation stage and analyzed in transmission geometry. The incident beam was scanned and rastered to optimize orientation statistics. A beam-stop was used to minimize air scatter from the incident beam at low angles. Diffraction patterns were collected using a Hi-Star area detector located 15 em from the sample and processed using GADDS. The intensity in the GADDS image of the diffraction pattern was integrated using a step size of 0.04 °2θ. The integrated patterns display diffraction intensity as a function of 28. Prior to the analysis a silicon standard was analyzed to verify the Si 111 peak position.

### Example 1

### Preparation of Form D and Form E

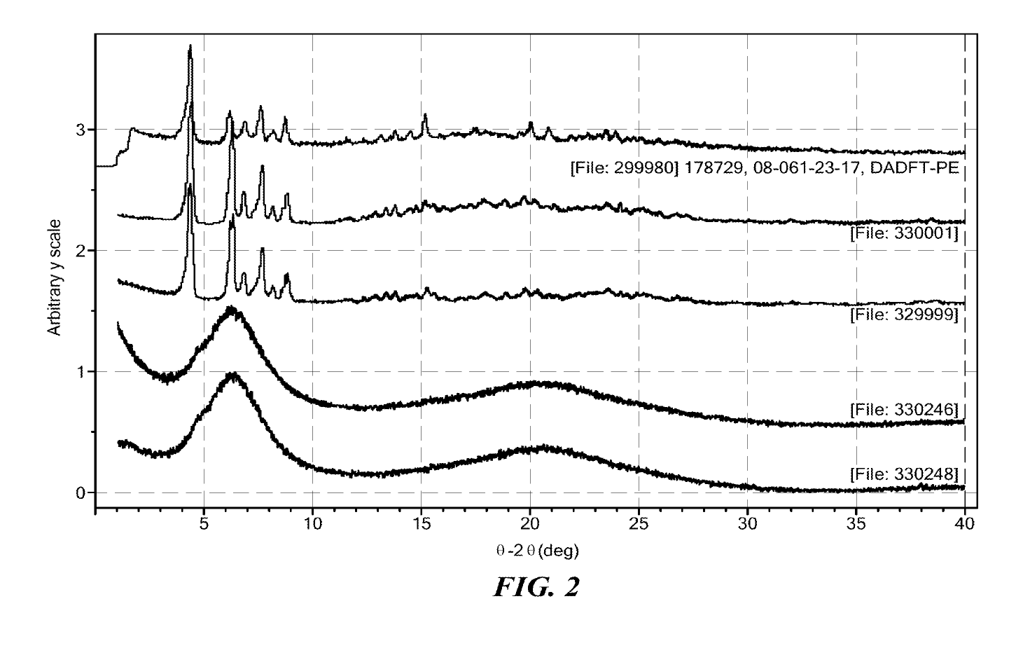

To obtain Form E, amorphous material was slurried in EtOH/H₂O/MTBE in a 5.8:1:20 ratio, vacuum filtered or centrifuged to obtain the solid form, and analyzed by XRPD and Karl-Fischer both wet and after overnight vacuum drying at approximately 53° C. The XRPD and Karl-Fischer analyses identified both samples as Form E. The water content of filtered and centrifuged samples were 7.99 and 7.00% (w/w), respectively (see Table 1). Form E was obtained directly from the process solvents isolated as a moist cake. The centrifugation of the moist cake, to remove excess solvent, does not affect the crystalline form. See FIGS. 1a, 1b, and 2.

TABLE 1

| Conditions | Water content (wt %) | XRPD Result |
|---|---|---|
| Vacuum filtered slurry, | 7.99 | E |
| wet | | |
| Vacuum filtered slurry, | 1.00 | Amorphous |
| vacuum/53° C., overnight | | |
| Centrifuged slurry, wet | 7.00 | E |
| Centrifuged slurry, | 0.86 | Amorphous |
| vacuum/53° C., overnight | | |

As described above, Form D was obtained via DVS experimentation with Form B, and Form B was generated with crystallization experiments using solvent mixtures containing methanol or water (e.g., MeOH/acetone, IPA/MeOH, heptane/MeOH, water (solvent grinding), water (antisolvent precipitation), or water (slurry at ambient)). Dynamic vapor sorption/desorption (DVS) data were collected on a VTI SGA-100 Vapor Sorption Analyzer. Sorption and desorption data were collected over a range of 5% to 95% relative humidity (RH) at 10% RH intervals under a nitrogen purge. Samples were not dried prior to analysis. Equilibrium criteria used for analysis were less than 0.0100% weight change in 5 minutes, with a maximum equilibration time of 3 hours if the weight criterion was not met. Data were not corrected for the initial moisture content of the sample. Sodium chloride and polyvinylpyrrolidine were used as calibration standards. See FIG. 3.

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

1. A solid form of Compound 1:

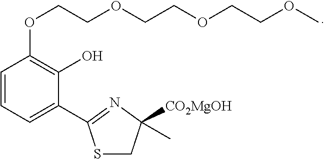

2. The solid form of claim 1, wherein the solid form is crystalline.

3. The solid form of claim 3, wherein the solid form is polymorph Form D.

4. The solid form of claim 3, wherein the solid form is polymorph Form E.

5. The solid form of claim 3, having one or more peaks in its X-ray powder diffraction pattern selected from those at about 3.1, about 6.4, about 6.9, and about 18.3 degrees 2-theta.

6. The solid form of claim 5, having two or more peaks in its X-ray powder diffraction pattern selected from those at about 3.1, about 6.4, about 6.9, and about 18.3 degrees 2-theta.

7. The solid form of claim 6, having three or more peaks in its X-ray powder diffraction pattern selected from those at about 3.1, about 6.4, about 6.9, and about 18.3 degrees 2-theta.

8. The solid form of claim 7, having substantially all of the peaks in its X-ray powder diffraction pattern selected from those at about 3.1, about 6.4, about 6.9, and about 18.3 degrees 2-theta.

9. The solid form of claim 4, having one or more peaks in its X-ray powder diffraction pattern selected from those at about 4.1, about 6.0, about 6.1, about 6.9, about 7.5 and about 8.9 degrees 2-theta.

10. The solid form of claim 9, having two or more peaks in its X-ray powder diffraction pattern selected from those at about 4.1, about 6.0, about 6.1, about 6.9, about 7.5 and about 8.9 degrees 2-theta.

11. The solid form of claim 10, having three or more peaks in its X-ray powder diffraction pattern selected from those at about 4.1, about 6.0, about 6.1, about 6.9, about 7.5 and about 8.9 degrees 2-theta.

12. The solid form of claim 11, having substantially all of the peaks in its X-ray powder diffraction pattern selected from those at about 4.1, about 6.0, about 6.1, about 6.9, about 7.5 and about 8.9 degrees 2-theta.

13. The solid form of any preceding claim, wherein said compound is substantially free of impurities.

14. A composition comprising the solid form of any preceding claim, and a pharmaceutically acceptable carrier or excipient.

15. A method for treating metal overload in a subject in need of treatment thereof, comprising the step of administering to the subject a therapeutically effective amount of Compound 1.

16. The method of claim 15, wherein Compound 1 is administered at a daily dose ranging from 10-250 mg/kg of body weight.

17. The method of claim 15, wherein Compound 1 is administered at a daily dose ranging from 40-80 mg/kg of body weight.

18. The method of claim 15, wherein the method results in no substantial adverse effects.

19. The method of claim 15, wherein the metal overload is uranium overload.

20. The method of claim 15, wherein the metal overload is iron overload.

21. The method of claim 20, wherein the iron overload is transfusional iron overload.

22. The method of claim 20, wherein the iron overload is caused by increased iron absorption.

23. The method of claim 15, wherein the subject is suffering from β-thalassemia-intermediate, β-thalassemia-major, non-transfusion dependent Thalassaemia (NTDT), Blackfan-Diamond anemia, Sideroblastic anemia, sickle cell disease, aplastic anemia, red cell aplasia, Myelodysplasia (MDS), chronic myelofibrosis, paroxysmal nocturnal hemoglobinuria, off-therapy leukemia, hereditary hemochromatosis, or porphyria cutanea tarda.

24. The method of claim 23, wherein the subject is suffering from β-thalassemia-intermediate.

25. The method of claim 23, wherein the subject is suffering from β-thalassemia-major

26. The method of claim 23, wherein the subject is suffering from sickle cell disease.

27. The method of claim 23, wherein the subject is suffering from Myelodysplasia (MDS).

28. The method of claim 15, wherein the therapeutically effective amount of Compound 1 is a capsule or tablet.

29. The method of claim 15, wherein the subject is an adult.

30. The method of claim 15, wherein the subject is a pediatric patient.
